(19) United States
(12) Patent Application Publication
(10) Pub. No.: US 2023/0158147 A1
(43) Pub. Date: May 25, 2023

(54) WATER SOLUBLE FORMULATIONS CONTAINING CANNABINOIDS

(71) Applicants: National Research Council of Canada, Ottawa (CA); Next Remedies Inc., Toronto (CA)

(72) Inventors: Anja Vogt, Charlottetown (CA); Martin Elia, Brackley Beach (CA); Bob Chapman, Stratford (CA); Jorge Henrique Santos, Toronto (CA); Jose Gabriel Gomes Jardim, Marina Del Rey, CA (US)

(21) Appl. No.: 17/919,147

(22) PCT Filed: Apr. 16, 2021
(86) PCT No.: PCT/CA2021/050516

§ 371 Date: Oct. 14, 2022

(30) Foreign Application Priority Data
Apr. 16, 2020 (CA) 3078970

(51) Int. Cl. A61K 47/22; A61K 31/352; A61K 31/05; A61K 47/14; A61K 9/107

(52) U.S. Cl. CPC A61K 47/22; A61K 31/352; A61K 31/05; A61K 47/14; A61K 9/1075

(57) ABSTRACT

The present disclosure relates to water-soluble formulations containing cannabinoids. In particular, the formulations comprise at least one isolated cannabinoid, cannabinoid oil or cannabinoid resin, and a solubilizing agent of Formula (I), where the group X is a residue of a hydrophobic moiety selected from sterols, tocopherols, and derivatives thereof, and the group Y is a residue of a hydrophilic moiety selected from polyalcohols, polyethers, and derivatives thereof. In preferred embodiments, the cannabinoid comprises cannabidiol (CBD) or Δ9-tetrahydrocannabinol (THC) and the hydrophobic moiety is polyoxyethanyl-α-tocopheryl sebacate (TPS). In embodiments, the formulations comprise a carrier oil, such as a medium length triglyceride (MCT), and are dispersed in water in the form of micelles.

### CROSS-REFERENCE TO RELATED APPLICATIONS

The application claims the benefit of priority to Canadian Patent Application No. 3,078,970, filed Apr. 16, 2020, the contents of which is incorporated herein by reference in its entirety.

### FIELD

The present disclosure relates, for example, to water-soluble formulations containing cannabinoids. In particular, the formulations comprise a cannabinoid composition, such as an isolated cannabinoid, cannabinoid oil, or cannabinoid resin, and a solubilizing agent.

### INTRODUCTION

Cannabinoids are a diverse class of chemical substances that act on the cannabinoid receptors. Phytocannabinoids can be found in various plants, but are most common in Cannabis sativa species, known as Cannabis and Hemp plants. While over 100 different cannabinoids have been isolated from these plants, the two most notable phytocannabinoids are Δ9-Tetrahydrocannabinol (THC) and Cannabidiol (CBD).

THC is the main psychoactive compound in Cannabis that is a partial agonist at the cannabinoid receptors CB1 and CB2. CBD is non-psychotropic and is under discussion for various effects against neurodegeneration and mental disorders, e.g. against epilepsy, pain, anxiety. It has a low affinity as antagonist of CB1 and CB2 cannabinoid receptors. THC and CBD are stored as tetrahydrocannabinolic acid (THCA) and Cannabidiolic acid (CBDA) in the plant and are decarboxylated over time or under heat to THC and CBD. In plant material intended for smoking, the conversion can take place during the smoking process, but materials for ingestion, such as oils and edibles, need to be heated to release free THC and CBD.

Bioavailability of cannabinoids though inhalation is high and this is the most efficient and rapid delivery route. Peak concentrations are reached rapidly, within 10 minutes, though a wide range of intra- and interpersonal variability exists, partly due to differences in holding times and inhalation volumes. Bioavailability ranges from 2% to 56% (average 30%) for THC and 11% to 45% (average 31%) for CBD.

The oral bioavailability of cannabinoids is much lower (between 4% and 20% for THC and around 6% for CBD) and slower than inhalation bioavailability due to several factors. First, cannabinoids have a very low water solubility, which restricts their bioavailability. Second, absorption from the gastrointestinal system varies greatly from person to person and is influenced by different conditions, such as food intake. Third, cannabinoids are degraded in the stomach, with the degree of degradation depending on gastric pH and food intake. Fourth, cannabinoids are subjected to a significant first-pass metabolism in the liver by cytochrome P450 enzymes, resulting in active and inactive metabolites. These factors make the rate and speed of absorption hard to predict and make oral dosing for a desired pharmacological effect unreliable. Additionally, tₘₐₓ, the time to reach peak plasma concentration, is high (on the order of hours) for oral administration of oil soluble formulations of cannabinoids like THC and CBD. This can lead to overdosing by consumers who may be inclined to take additional doses before the effect of the first dose sets in.

More than 100 metabolites of cannabinoids have been identified. Two of the main metabolites of THC are 11-hydroxy-Δ9-tetrahydrocannabinol (11-OH-THC), which is psychoactive, and 1-nor-Δ9-tetrahydrocannabinol-9-carboxylic acid (9-COOH-THC), which is not psychoactive. Two of the main metabolites of CBD are 7-hydroxy-CBD (7-OH-CBD) and (3R-trans)-cannabidiol-7-oic acid (7-COOH-CBD). Conversion of CBD to THC has been reported but is not conclusively demonstrated in vivo.

Oils derived from cannabis plant materials are often commercially extracted using either supercritical CO₂ extraction or ethanol. The extraction products are very viscous, dark brown resins with high concentrations of cannabinoids and varying levels of terpenoids. Typically, the resins are diluted with other oils like MCT oil or hemp oil for consumer use.

### SUMMARY

The present disclosure is directed to water-soluble formulations comprising a cannabinoid composition and a solubilizing agent. In embodiments, the cannabinoid composition comprises an isolated cannabinoid, a cannabinoid oil, or a cannabinoid resin. In one embodiment, the formulations are formulated into emulsions which are stable for long time periods, and yet contain high concentrations of cannabinoids, and upon administration, result in increased oral bioavailability including high maximum plasma concentrations, short times to reach maximum plasma concentration, and high total drug exposure (AUC).

In one embodiment, the present disclosure includes a formulation comprising:
(i) a cannabinoid composition; and
(ii) at least one compound of Formula (I):

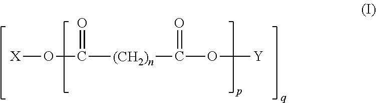

wherein
   X is a residue of a hydrophobic moiety selected from sterols, tocopherols and derivatives thereof;
   Y is a residue of a hydrophilic moiety selected from polyalcohols, polyethers and derivatives thereof;
   n is an integer of from 0 to 18;
   p is 1 or 2; and
   q is 1 or 2.

In an embodiment, the cannabinoid composition comprises an isolated cannabinoid.

In another embodiment, the cannabinoid composition comprises a cannabinoid oil or resin.

In another embodiment, the formulations further comprise a carrier oil.

In one embodiment, the water-soluble formulations are mixed with water to form emulsions or nano-emulsions.

The present disclosure also includes a pharmaceutical or cosmetic formulation comprising an emulsion of the present disclosure and a biologically acceptable liquid carrier; and a nutraceutical formulation or dietary supplement comprising an emulsion of the present disclosure and a biologically acceptable liquid carrier.

The present disclosure also includes a beverage or beverage enhancer comprising the emulsion of the present disclosure.

The present disclosure also includes a use of an emulsion of the present disclosure for the preparation of a pharmaceutical formulation or a cosmetic formulation; a use of an emulsion of the present disclosure for the preparation of a nutraceutical formulation or dietary supplement; and a use of an emulsion of the present disclosure for the preparation of a beverage or beverage enhancer.

The present disclosure also includes a method for preparing an emulsion, the method comprising:
heating a water-soluble formulation of the present disclosure to form a homogeneous melt; and
combining the homogeneous melt with water to obtain the emulsion.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The present disclosure will now be described in greater detail with reference to the drawings in which:

FIG. 1 shows dynamic light scattering graphs of formulations of the present disclosure: PTS-MCT-THC (A) and PTS-MCT-CBD (right);

FIG. 2 are photos demonstrating the stability of formulations after 12 weeks storage at 4° C. (B) and at 32° C. (A), the latter being equivalent to 52 weeks shelf-life at room temperature;

FIG. 3 shows a comparison of mean (n=5 or 6) plasma concentration versus time profiles of THC, following p.o. administration of PTS-MCT-THC or MCT-THC formulations in a rat model;

FIG. 4 shows a summary of cₘₐₓ, tₘₐₓ and AUCₒ calculated for THC and CBD determined following administration of THC or CBD formulations in a rat model; and

FIG. 5 shows plasma and brain concentrations of THC and CBD determined following administration of PTS-MCT-THC and PTS-MCT-CBD (n=3 or 6) formulations in a rat model.

### DESCRIPTION OF VARIOUS EMBODIMENTS

### (I) Definitions

Unless otherwise indicated, the definitions and embodiments described in this and other sections are intended to be applicable to all embodiments and aspects of the present application herein described for which they are suitable as would be understood by a person skilled in the art.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The term “and/or” as used herein means that the listed items are present, or used, individually or in combination. In effect, this term means that “at least one of” or “one or more” of the listed items is used or present.

As used in this application, the singular forms “a”, “an” and “the” include plural references unless the content clearly dictates otherwise.

The term “suitable” as used herein means that the selection of specific reagents or conditions will depend on the reaction being performed and the desired results, but nonetheless, can generally be made by a person skilled in the art once all relevant information is known.

The term “PTS monomer” as used herein refers to a compound having the following general structure:

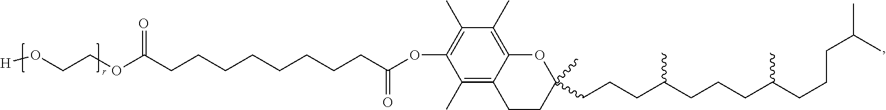

wherein r is 12 or 13.

The term “PTS dimer” as used herein refers to a compound having the following general structure:

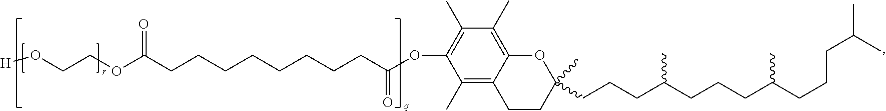

wherein r is 12 or 13; and q is 2.

The term “subject” as used herein includes all members of the animal kingdom including mammals, and suitably refers to humans.

The term “biologically acceptable” as used herein means compatible with the treatment of, administration to and/or use in subjects.

In embodiments of the application, the compounds described herein have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (e.g. less than 20%, suitably less than 10%, more suitably less than 5%) of compounds of the application having alternate stereochemistry.

The term “polyalcohol” as used herein refers a compound having the general formula HOCH₂(CHOH)ₓCH₂OH.

The term “polyether” as used herein refers to a compound that is an oligomer or polymer having repeating units comprising an ether functionality such as polyethylene glycol.

The term “cannabinoid composition” as used herein refers to a composition comprising at least one cannabinoid. The term “cannabinoid composition” includes, for example, an isolated cannabinoid, a cannabinoid oil, a cannabinoid resin, and multiples and/or mixtures thereof.

The term “cannabinoid oil or resin” as used herein refers to oils or resins derived from cannabis plant materials (for example, Cannabis sativa) which have been extracted with solvents, such as ethanol, or supercritical gases, such as carbon dioxide.

The term “isolated” as used herein refers to a molecule that has been been at least partially separated from the natural environment, e.g. from a cannabis plant. The term “isolated” is also used to refer to synthetic molecules. For example, an isolated cannabinoid may be at least partially separated from a plant, or it may be synthesized or semi-synthesized.

The term “carrier oil” as used herein refers to an oil which helps to dissolve, dilute, disperse or suspend the cannabinoid composition.

### (II) FORMULATIONS OF THE DISCLOSURE

The present disclosure is directed to water-soluble formulations comprising a cannabinoid composition and a solubilizing agent. In one embodiment, the formulations are formulated into emulsions which are stable for long time periods, and yet contain high concentrations of cannabinoids, and upon administration, result in increased oral bioavailability including high maximum plasma concentrations, short times to reach maximum plasma concentration, and high total drug exposure (AUC).

In one embodiment, the present disclosure includes a formulation comprising:
(i) a cannabinoid composition; and
(ii) at least one compound of Formula (I):

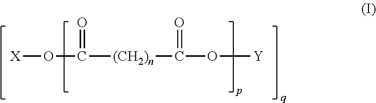

wherein
   X is a residue of a hydrophobic moiety selected from sterols, tocopherols and derivatives thereof;
   Y is a residue of a hydrophilic moiety selected from polyalcohols, polyethers and derivatives thereof;
   n is an integer of from 0 to 18;
   p is 1 or 2; and
   q is 1 or 2.

In an embodiment, the cannabinoid composition comprises or consists of an isolated cannabinoid.

In an embodiment, the cannabinoid composition comprises or consists of a cannabinoid oil or resin.

In an embodiment, the hydrophobic moiety in Formula (I) is selected from cholesterol, 7-dehydrocholesterol, campesterol, sitosterol, ergosterol, stigmasterol, α-tocopherol, β-tocopherol, γ-tocopherol and δ-tocopherol. In another embodiment, the hydrophobic moiety is α-tocopherol.

In an embodiment, the hydrophilic moiety in Formula (I) is a polyether. In another embodiment, the hydrophilic moiety is a polyalcohol. In an embodiment, the polyether is a polyalkylene glycol. In another embodiment, the polyalkylene glycol is a polyethylene glycol or a polypropylene glycol. In a further embodiment, the polyalkylene glycol is a polyethylene glycol. The term “polyalkylene glycol” as used herein includes polyalkylene glycols having an esterifiable hydroxy group at least at one end of the polymer as well as derivatives of such polymers having esterifiable carboxy groups. The residue of the hydrophilic moiety is the entire hydrophilic molecule, except for its esterified hydroxy or carboxy group or groups, such as a terminal hydroxy group of a polyethylene glycol. In an embodiment, the polyethylene glycol has an average molecular weight of from about 300 to about 5000. In another embodiment of the present application, the polyethylene glycol has an average molecular weight of from about 400 to about 1000.

In an embodiment, when p and q are equal to 1 and the hydrophobic moiety is cholesterol, n is greater than 4 and not equal to 8. In another embodiment of the present application, when p and q are equal to 1 and the hydrophobic moiety is α-(+)-tocopherol, n is not equal to 2.

In an embodiment, n is an integer of from 2 to 10. In another embodiment, n is an integer of from 6 to 10. In a further embodiment, n is 8.

In an embodiment, p is 1. In another embodiment, p is 2.

In an embodiment, q is 1. In another embodiment, q is 2.

In an embodiment, the at least one compound of Formula (I) is polyoxyethanyl-α-tocopheryl sebacate (PTS) monomer, PTS dimer, or a combination thereof. In another embodiment, the at least one compound of Formula (I) is PTS monomer. In a further embodiment, the at least one compound of Formula (I) is PTS dimer. In another embodiment, the at least one compound of Formula (I) is a combination of PTS monomer and PTS dimer.

In one embodiment, the formulation further comprises a carrier oil. In one embodiment, the carrier oil improves the stability of the emulsion. In one embodiment, the carrier oil is any suitable oil. For example, it would be appreciated by the person skilled in the art that when the composition is for use in subjects, the oil is a biologically acceptable oil. In an embodiment, the oil is hemp oil, sunflower oil, medium chain length triglyceride (MCT) oil, long chain triglycerides (LCT) oil, vegetable oil, soybean oil, olive oil or combinations of two or more thereof. In another embodiment, the oil is sunflower oil. In another embodiment, the oil is any biologically acceptable oil such as melatonin oil or ashwaganda oil. In a further embodiment, the oil is MCT oil or hemp oil.

In one embodiment, the ratio of the solubilizing agent (the compound of Formula (I)) to the cannabinoid composition (Formula (I):cannabinoid composition) (w/w) is from about 1:1 to about 15:1, or about 1:1 to about 10:1 or about 1:1 to 5:1, or about 2:1 or about 3:1. In embodiments where the formulations further comprise a carrier oil, the ratio of the solubilizing agent (the compound of Formula (I)) to the cannabinoid composition and the carrier oil (Formula (I):cannabinoid composition:carrier oil) (w/w) is from about 1:1:1 to about 15:1:1, or about 1:1:1 to about 10:1:1 or about 1:1:1 to 5:1:1, or about 2:1:1 or about 3:1:1.

In another embodiment, the ratio of the solubilizing agent (the compound of Formula (I)) to a mixture of the cannabinoid composition and the carrier oil (Formula (I):mixture) is from about 1:1 to about 15:1, or about 1:1 to about 10:1 or about 1:1 to 6:1, or about 2:1 or about 3:1. In embodiments, the mixture comprises:

about 1 to 99.9% carrier oil and about 99% to 0.1% cannabinoid oil, or about 10% to about 90% carrier oil and about 90% to about 10% cannabinoid oil, or about 25% to about 75% carrier oil and about 75% to about 25% cannabinoid oil, or about 40% to about 60% carrier oil and about 60% to about 40% cannabinoid oil, or about 50% carrier oil and about 50% cannabinoid oil; or
about 40% to about 99.9% carrier oil and about 60% to about 0.1% cannabinoid resin, or about 50% to about 75% carrier oil and about 50% to about 25% cannabinoid resin, or about 40% to 60% carrier oil and about 60% to about 40% cannabinoid resin, or about 50% carrier oil and about 50% cannabinoid resin; or about 25% to about 99.9% carrier oil and about 75 to about 0.1% cannabinoid, or about or about 50% to about 75% carrier oil and about 50% to about 25% cannabinoid.

In one embodiment, the formulations of the present disclosure contain at least about 10 mg/ml of THC or at least about 12.5 mg/ml of CBD, or from greater than 0 mg/ml to about 50 mg/ml of THC, CBD, or a combination thereof.

In one embodiment, the emulsions of the present disclosure are clear and stable emulsions (stable for at least 12 months at room temperature), and are water soluble solutions with a micelle size less than 20 nm.

The present disclosure also includes an emulsion comprising a composition of the present disclosure dispersed in water. It will be appreciated by a person skilled in the art that embodiments relating to the compositions in the emulsions of the present disclosure can be varied as described herein in relation to the compositions of the present disclosure.

In an embodiment, the composition is dispersed in the water in the form of micelles.

In an embodiment, the emulsion further comprises one or more additives. In another embodiment, the additives are selected from adjuvants, colorants, flavoring agents, preservatives, buffers and combinations thereof.

The present disclosure also includes a pharmaceutical or cosmetic formulation comprising an emulsion of the present disclosure and a biologically acceptable carrier. The pharmaceutical or cosmetic formulation may be formulated to allow delivery by any suitable route, including ingestion, topical delivery, buccal delivery, and sublingual delivery. The present disclosure further includes a nutraceutical formulation or dietary supplement comprising an emulsion of the present disclosure and a biologically acceptable carrier. The emulsions of the present disclosure may advantageously provide a format for subjects that does not involve swallowing pills but instead offers a solution for the administration or use of cannabinoids in an aqueous emulsion format or cream. Accordingly, the present disclosure also includes a pharmaceutical or cosmetic formulation comprising an emulsion of the present disclosure and a biologically acceptable liquid carrier as well as a nutraceutical formulation or dietary supplement comprising an emulsion of the present disclosure and a biologically acceptable liquid carrier.

In an embodiment, the pharmaceutical or cosmetic formulation is in the form of a spray, liquid, syrup or drop. In another embodiment of the present disclosure, the nutraceutical formulation or dietary supplement is in the form of a spray, liquid, syrup or drop. A person skilled in the art would know how to prepare suitable formulations.

The present application also includes a beverage or beverage enhancer comprising the emulsion of the present application.

The present application also includes a use of an emulsion of the present application for the preparation of a pharmaceutical formulation or a cosmetic formulation. The present application further includes a use of an emulsion of the present application for the preparation of a nutraceutical formulation or dietary supplement. The present application also includes a use of an emulsion of the present application for the preparation of a beverage or beverage enhancer. For example, an emulsion of the present application can be added to any suitable beverage base such as water. In an embodiment, from about 1 ml to about 10 ml, from about 1 mL to about 3 ml, or about 2 ml of the emulsion is added per every 250 ml of the beverage base such as water.

### (III) METHODS OF PREPARATION

In one embodiment, the water-soluble formulations are used to form stable emulsions having an average micelle size of less than about 20 nm, or between about 7.5 to about 20.0 nm or between about 9.5 to about 13.0 nm or about 12.0 nm, and are prepared by heating a formulation comprising a cannabinoid oil or resin, a carrier oil and a compound of Formula (I) (for example, the solubilizing agent PTS) to form a homogeneous melt and combining the homogeneous melt with water using either blending or high shear mixing, optional homogenization by use of a microfluidizer and rapid cool down using cooling, ice, cold water, or a mixture of ice and water to obtain the emulsion. In other embodiments, the average micelle size is less than about 20 nm, which can include sizes of 22 nm or 24 nm. In another embodiment, the formulations contain micelles having two different average sizes: an average size of about 12.0 nm, and an average size of about 40 nm or higher.

Accordingly, the present application also includes a method for preparing an emulsion, the method comprising:
heating a formulation of the present application to form a homogeneous melt, and
combining the homogeneous melt with water to obtain the emulsion.

It will be appreciated by a person skilled in the art that embodiments relating to the compositions in the methods for preparing an emulsion of the present application can be varied as described herein in relation to the compositions of the present application.

In an embodiment, the combining step comprises mixing the homogeneous melt and water at a temperature of from about 40° C. to about 95° C., for example, a temperature of from about 75° C. to about 80° C. for a time of about 15 minutes to about 4 hours, or about 15 minutes to about 60 minutes, or about 30 minutes.

In one embodiment, the combining step comprises mixing the homogeneous melt with water using either blending or high shear mixing, optionally followed by homogenization by use of a microfluidizer, and rapid cool down, using cooling, ice, cold water, or a mixture of ice and water to obtain the emulsion

In some embodiments, subsequent to mixing, the method further comprises processing the mixture through a microfluidizer. The conditions for processing the mixture through a microfluidizer are any suitable conditions. In an embodiment, the conditions comprise a single pass through the microfluidizer. In another embodiment, the conditions comprise a pressure of from about 10,000 psi to about 26,000 psi, or from about 10,000 psi to about 20,000 psi.

In another embodiment, the mixture is passed through a filter having a pore size of about 0.2 μm.

In an embodiment, the method further comprises cooling the mixture. It will be appreciated by the person skilled in the art that in embodiments comprising processing the mixture through a microfluidizer, the cooling can be subsequent or simultaneous to the processing of the mixture through the microfluidizer. The mixture is cooled to any suitable temperature. In an embodiment, the mixture is cooled to a temperature of about 10° C. to about 15° C. or about 4° C. In one embodiment, the mixture is cooled using ice.

In some embodiments, the cooling comprises mixing the homogeneous emulsion with ice or a combination of water and ice. In an embodiment, the ratio by volume of water:ice in the final combination is about 2:1. In other embodiments, the mixture is cooled using a cooling system.

Although the disclosure has been described in conjunction with specific embodiments thereof, if is evident that many alternatives, modifications and variations will be apparent to those skilled in the art. Accordingly, the disclosure is intended to embrace all such alternatives, modifications and variations that fall within the spirit and broad scope of the appended claims. In addition, citation or identification of any reference in this disclosure shall not be construed as an admission that such reference is available as prior art to the present disclosure.

### EXAMPLES

The operation of the disclosure is illustrated by the following representative examples. As is apparent to those skilled in the art, many of the details of the examples may be changed while still practicing the disclosure described herein.

### Example 1—Preparation of Formulations

Cannabis resins with high concentrations of cannabinoids were used to prepare water-soluble emulsions with Polyoxyethanyl-α-tocopheryl sebacate (PTS). Two different resins were used, one resin high in THC (84% THC), the other high in CBD (82% CBD and 1.6% THC). Both resins contained small amounts of minor cannabinoids as shown in Table 1 using the average of two analytical injections. Cannabinol (CBN), Cannabigerol (CBG), tetrahydrocannabinolic acid (THCA-A) and Cannabigerolic acid (CBGA) were not detected in any of the samples, while (Δ9-Tetrahydrocannabinol (THC), Cannabidiol (CBD), cannabichromine (CBC), Cannabidiolic acid (CBDA) and tetrahydrocannabivarin (THCV)) were observed in various amounts in the formulated PTS preparations. To formulate the PTS emulsions, the cannabis resins were diluted with MCT oil (medium-chain triglyceride oil) in a ratio of 50% resin to 50% MCT oil. For the concentrated cannabis resins used, the addition of carrier oil was found to be necessary to formulate the PTS emulsions. However, the addition of a carrier oil is not expected to be necessary for formulation of all cannabis extracts or products. For example, less viscous concentrates or liquid extracts, such as cannabis oil, may not require the addition of a carrier oil.

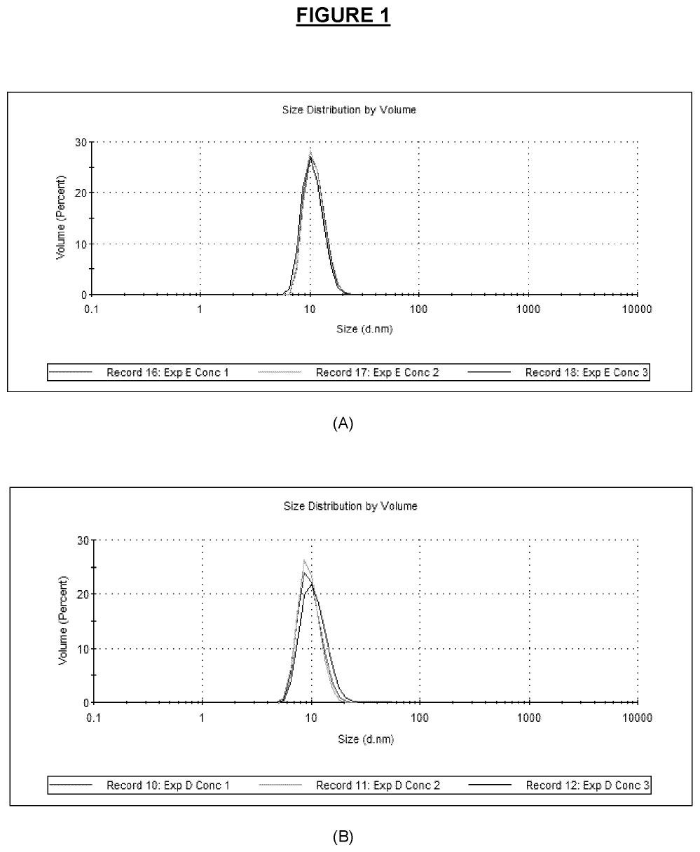

To formulate the emulsions, 4.4 g resin, 53 g PTS and 4.4 g MCT oil were mixed and warmed under stirring into a melt. Water was added and the mixture was stirred under heating to 76° C. for 30 minutes. The resulting solutions were processed through a microfluidizer at 10,000 to 20,000 psi and filtered through a 0.2 μm filter. Stock solution prepared contain 10.5 mg THC per ml (PTS-MCT-THC) or 12.5 mg CBD per ml (PTS-MCT-CBD) (see table 1). They are clear, yellow solutions (189 NTU and 192 NTU, respectively) with a micelle size between 9.5 nm and 11.0 nm (determined by DLS) (see FIG. 1). 10.53±0.01 mg THC per ml, average size 11.01±0.23 nm and PTS-MCT-CBD (right) 12.54±0.16 mg CBD per ml, average size 10.12±0.53 nm

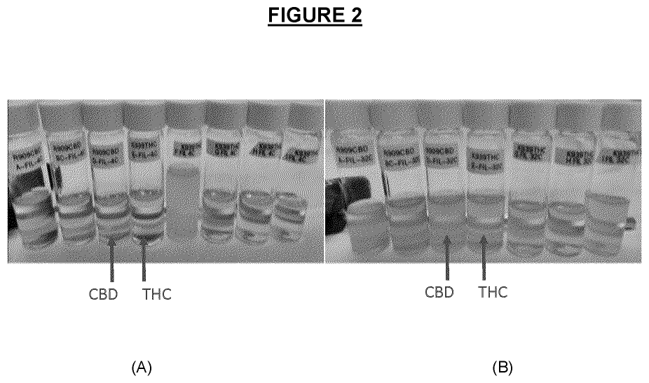

The formulations were monitored for their stability under accelerated shelf life stability conditions. They were stored at 32° C. and checked for every 2 weeks for 12 weeks. The factor to compare accelerated shelf life testing at 32° C. to stability at room temperature has been previously determined to be 4 (i.e. 1 week at 32° C. equals 1 month at room temperature). The solutions were visually observed for clarity and colour, as well as precipitates or phase separations. The formulations were stable for a minimum of 12 weeks at 32° C., which is equivalent to 12 months at room temperature. No significant clouding, precipitation or separation was observed as shown in FIG. 2.

### Example 2—PK Rat Study

Samples of the emulsions of Example 1 were used in a PK rat study compared to solutions of THC and CBD resin in MCT oil.

A PK Bioavailability Study on male Sprague-Dawley rats to compare the bioavailability of the PTS-MCT-THC and PTS-MCT-CBD emulsions to MCT-THC and MCT-CBD solutions. A single oral dose of 20 mg THC or CBD per kg BW was administered to 6 rats in each group. The concentrations of (THC), (CBD) and 4 metabolites (11-hydroxy-Δ9-tetrahydrocannabinol (11-OH-THC), 11-nor-Δ9-tetrahydrocannabinol-9-carboxylic acid (9-COOH-THC), 7-hydroxy-CBD (7-OH-CBD) and (3R-trans)-cannabidiol-7-oic acid (7-COOH-CBD) in blood plasma were monitored at nine time-points for up to 24 h. Terminal brain samples were collected from the animals following the 24 h plasma collection. Two additional satellite groups of 9 rats were orally dosed with PTS-MCT-THC and PTS-MCT-CBD. Terminal blood and brain were collected at 1, 2 and 4 hours post-dose from each satellite group (3 rats per time-point). Brain and plasma samples were analysed using tandem liquid chromatography-mass spectrometry (LC-MS/MS).

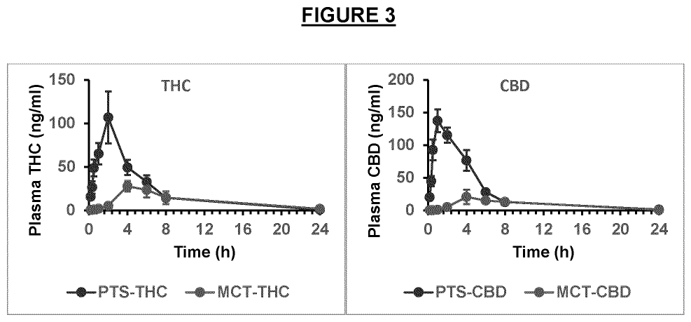

The maximum plasma concentration is increased in the PTS group compared to the MCT oil group for both THC (3.9 times, 107±72.9 ng/ml vs. 27.7±13.6 ng/ml) and CBD (6.7 times, 137±42.7 ng/ml vs. 20.6±27.1 ng/ml) and is reached faster for PTS containing THC formulations compared to MCT oil formulations (2 h compared to 4 h) and for PTS containing CBD formulations compared to MCT oil formulations (1 h compared to 4 h) as shown in FIG. 3.

Both 11-OH-THC and 9-COOH-THC were detected in plasma samples after PTS-MCT-THC and MCT-THC treatment. Administration of PTS-MCT-THC results in higher plasma concentrations of THC and these two metabolites with the cₘₐₓ being 3.2-, 2.9- and 2.0 times higher for PTS-MCT-THC compared to MCT-THC, respectively, in a shorter time (tₘₐₓ 50-60% shorter).

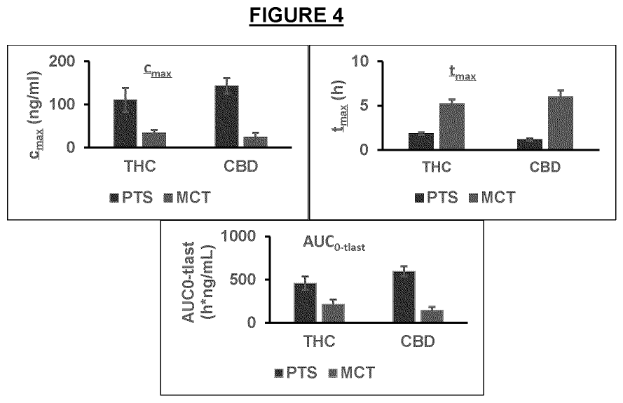

7-COOH-CBD, as well as THC, 11-OH-THC and 9-COOH-THC (due to THC content in CBD oil) were detected in plasma samples after both PTS-MCT-CBD and MCT-CBD administration, but no 7-OH-CBD was observed. Administration of PTS-MCT-CBD compared to MCT-CBD resulted in higher plasma concentrations (cₘₐₓ) of CBD and 7-COOH-CBD (6.0- and 5.1 times higher, respectively) in a shorter time (tₘₐₓ, 70-80% shorter), as shown in FIG. 4.

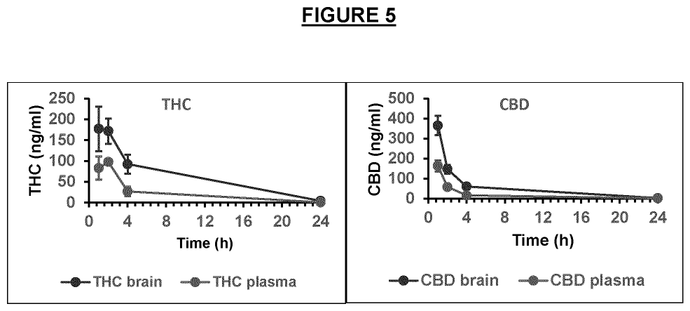

The maximum observed concentration in plasma (cₘₐₓ) is 3.2 times higher for PTS-MCT-THC formulations compared to MCT-THC solutions (110±69.3 ng/ml vs. 34.2±15.8 ng/ml) and 6.0 times higher for PTS-MCT-CBD formulations compared to MCT-CBD solutions (143±44.1 ng/ml vs. 23.8±25.9 ng/ml). The time to reach cₘₐₓ in plasma (tₘₐₓ) is 2.8 times shorter for PTS-MCT-THC emulsions compared to MCT-THC solutions (1.83 h±0.408 h vs. 5.20 h±1.10 h) and 5.0 times shorter for PTS-MCT-CBD emulsions compared to MCT-CBD solutions (1.17 h±0.408 h vs. 6.00 h±1.79 h). The area under the concentration vs time curve (AUC₀₋ₗₐₛₜ) representing the total drug exposure is 2.2× times higher for PTS-MCT-THC formulations (459±197 h*ng/mL vs. 212±128 h*ng/mL) and 4.2 times higher for PTS-MCT-CBD formulations (596±146 h*ng/mL vs. 141±102 h*ng/mL, as shown in FIG. 5.

THC and its metabolites can be detected in all brain samples. Brain concentrations of THC and 11-OH-THC exceed plasma concentrations at 1, 2 and 4 hours post-dose by 1.75 to 4.2 times for THC and 6.7 to 8.3 times for 11-OH-THC. Brain concentrations of 7-COOH-THC are lower than plasma concentrations.

Brain concentrations of CBD exceed plasma concentrations at 1, 2 and 4 hours post-dose by 2.3 to 4.0 times. Brain concentrations of 7-COOH-CBD are very low.

In summary, PTS can be used to form stable emulsions of cannabis oils high in THC and/or high in CBD, in concentrations of at least 10 mg/ml. Other prepared emulsions contain 5 mg/mL, 15 mg/mL, 20 mg/mL, and 30 mg/ml of THC and/or CBD, as well as other terpenes. The nanoemulsions are stable, clear solutions with an average micelle size under 20 nm. The bioavailability of formulations comprising PTS and cannabis oil is significantly and surprisingly improved and faster after oral ingestion in a rat pK model compared to MCT oil solutions of cannabis oils that lack PTS.

While the present disclosure has been described with reference to what are presently considered to be the preferred examples, it is to be understood that the disclosure is not limited to the examples described herein. To the contrary, the present disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

TABLE 1
Summary of cannabinoid concentrations determined by LC-HRMS in μg/mL

| | Sample | CBD | THC | CBC | CBDA | THCV |
|---|---|---|---|---|---|---|
| resin | CBD Resin | 819 ± 6 | 16 ± 0.7 | 36 ± 2 | 63 ± 0.3 | n.d. |
| | (R909CBD resin) | | | | | |
| | THC Resin | n.d. | 874 ± 3 | 14 ± 1 | n.d. | — |
| | (SATIVA resin) | | | | | |
| PTS | PTS-MCT-CBD | 12542 ± 161 | 332 ± 7 | 602 ± 2 | 869 ± 6 | n.d. |
| formulation | (R909CBD) | | | | | |
| | PTS-MCT-THC | n.d. | 10528 ± 8 | 156 ± 1 | n.d. | 57 ± 3 |
| | (K939THC) | | | | | |

*Resins were weighed and dissolved in methanol/IPA as 1 mg/mL

1. A formulation comprising:
(i) a cannabinoid composition; and
(ii) at least one compound of Formula (I): 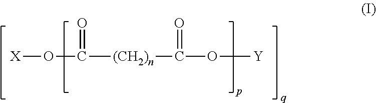
wherein
X is a residue of a hydrophobic moiety selected from sterols, tocopherols and derivatives thereof;
Y is a residue of a hydrophilic moiety selected from polyalcohols, polyethers and derivatives thereof;
n is an integer of from 0 to 18;
p is 1 or 2; and
q is 1 or 2.

2. The formulation of claim 1, wherein the cannabinoid composition comprises an isolated cannabinoid.

3. The formulation of claim 1, wherein the cannabinoid composition comprises a cannabinoid oil or cannabinoid resin.

4. The formulation of claim 1, wherein the hydrophobic moiety in Formula (I) is selected from cholesterol, 7-dehydrocholesterol, campesterol, sitosterol, ergosterol, stigmasterol, α-tocopherol, β-tocopherol, γ-tocopherol and δ-tocopherol.

5. The formulation of claim 1, wherein the hydrophobic moiety is α-tocopherol.

6. The formulation of claim 1, wherein the hydrophilic moiety in Formula (I) is a polyether.

7. The formulation of claim 6, wherein the polyether is a polyalkylene glycol.

8. The formulation of claim 7, wherein the polyalkylene glycol is a polyethylene glycol or a polypropylene glycol.

9. The formulation of claim 8, wherein the polyethylene glycol has an average molecular weight of from about 300 to about 5000, or about 400 to about 1000.

10. The formulation of claim 1, wherein the hydrophilic moiety in Formula (I) is a polyalcohol.

11. The formulation of claim 1, wherein when p, m and q are all equal to 1 and the hydrophobic moiety is cholesterol, n is greater than 4 and not equal to 8.

12. The formulation of claim 1, wherein when p and q are equal to 1, the hydrophobic moiety is α-(+)-tocopherol, and n is not equal to 2.

13. The formulation of claim 1, wherein n is an integer of from 2 to 10, or 6 to 10, or 8.

14. The formulation of claim 1, wherein the at least one compound of Formula (I) is polyoxyethanyl-α-tocopheryl sebacate (PTS) monomer, PTS dimer or a combination thereof.

15. The formulation of claim 14, wherein the at least one compound of Formula (I) is PTS monomer.

16. The formulation of claim 14, wherein the at least one compound of Formula (I) is PTS dimer.

17. The formulation of claim 14, wherein the at least one compound of Formula (I) is a combination of PTS monomer and PTS dimer.

18. The formulation of claim 1, wherein the formulation further comprises a carrier oil which is a biologically acceptable oil.

19. The formulation of claim 18, wherein the biologically acceptable oil is hemp oil, sunflower oil, medium chain length triglyceride (MCT) oil, long chain triglycerides (LCT) oil, vegetable oil, soybean oil, olive oil or a combination of any two or more thereof.

20. The formulation of claim 19, wherein the biologically acceptable oil is hemp oil or medium chain length triglyceride (MCT) oil.

21. The formulation of claim 1, wherein the ratio of the compound of Formula (I) to the cannabinoid composition (w/w) is from about 1:1 to about 15:1, or about 1:1 to about 10:1, or about 1:1 to 5:1, or about 2:1, or about 3:1.

22. The formulation of claim 18, wherein the ratio of the compound of Formula (I) to the cannabinoid composition and the carrier oil (Formula (I):cannabinoid composition:carrier oil) (w/w) is from about 1:1:1 to about 15:1:1, or about 1:1:1 to about 12:1:1 or about 1:1:1 to 5:1:1, or about 2:1:1 or about 3:1:1.

23. The formulation of claim 18, wherein the ratio of the compound of Formula (I) to a mixture of the cannabinoid composition and the carrier oil (Formula (I):mixture) (w/w) is from is from about 1:1 to about 15:1, or about 1:1 to about 10:1, or about 1:1 to 6:1, or about 2:1 or about 3:1.

24. The formulation of claim 1, wherein the formulation comprises at least about 10 mg/ml of THC or at least about 12.5 mg/ml of CBD.

25. An emulsion comprising the formulation as defined in claim 1 dispersed in water.

26. The emulsion of claim 25, wherein the formulation is dispersed in the water in the form of micelles.

27. The emulsion of claim 26, wherein the micelles have an average size of less than about 20 nm.

28. The emulsion of claim 25, further comprising at least one adjuvant, colorant, flavoring agent, preservative, or buffer, or comprising a combination of any two or more thereof.
